(19) United States
(12) Patent Application Publication
(10) Pub. No.: US 2013/0199313 A1
(43) Pub. Date: Aug. 8, 2013

(54) METHOD FOR EVALUATING THE SOLUBILITY OF A CRYSTALLINE SUBSTANCE IN A POLYMER

(71) Applicants: Geoff G. Zhang, Libertyville, IL (US); Lian Yu, Madison, WI (US); Jing Tao, Madison, WI (US); Markus Maegerlein, Mannheim (DE)

(72) Inventors: Geoff G. Zhang, Libertyville, IL (US); Lian Yu, Madison, WI (US); Jing Tao, Madison, WI (US); Ye Sun, Madison, WI (US); Markus Maegerlein, Mannheim (DE)

(73) Assignees: Abbott GmbH & Co, KGW, Wiesbaden (DE); Boards of Regents of the University of Wisconsin System, Madison, WI (US); AbbVie Inc., North Chicago, IL (US)

(21) Appl. No.: 13/828,238

(22) Filed: Mar. 14, 2013

Related U.S. Application Data
(63) Continuation of application No. 13/522,654, now abandoned.
Application No. 13/522,654 is a continuation of application No. 61/297,424, filed on Jan. 22, 2010.

(51) Int. Cl. G01N 33/15

(52) U.S. Cl. CPC G01N 33/15
USPC 73/866

(57) ABSTRACT

A method for evaluating the solubility of a crystalline substance in a polymer comprises a) providing at least one sample of an intimate crystalline substance/polymer mixture at a predetermined crystalline substance concentration; b) annealing the sample at a constant temperature Tₐ for a period of time; c) heating the annealed sample while recording the heat flux over time by DSC; d) identifying a DSC dissolution endotherm in the recorded heat flux, if any; e) considering the crystalline substance concentration as a concentration above the crystalline substance solubility in the polymer at temperature Tₐ when there is a DSC dissolution endotherm identified, and considering the crystalline substance concentration as a concentration less than or equal to the crystalline substance solubility in the polymer at temperature Tₐ when there is no DSC dissolution endotherm identified. Thus, the method yields the upper and lower bounds for the equilibrium solubility at a given temperature or the upper and lower bounds for the equilibrium solubility temperature at a given crystalline substance concentration. The method improves accuracy of measurement near the glass transition temperature.

### RELATED APPLICATIONS

This application is a continuation of U.S. application Ser. No. 13/522,654, filed Jul. 17, 2012, pending, which is the U.S. national phase, pursuant to 35 U.S.C. §371, of International application Ser. No. PCT/EP2011/050792, filed Jan. 21, 2011, designating the United States and published in English on Jul. 28, 2011 as publication WO 2011/089202 A1, which claims priority to U.S. provisional application Ser. No. 61/297,424, filed Jan. 22, 2010. The entire contents of the aforementioned patent applications are incorporated herein by this reference.

### FIELD OF THE INVENTION

Pharmaceutical scientists increasingly face the challenge of delivering poorly water soluble drugs. To meet this challenge, attempts have been made to use amorphous solids in place of crystals in pharmaceutical formulations. Amorphous solids are preferred physical forms because they dissolve more rapidly than crystalline solids when contacted with a liquid medium such as gastric fluid. The ease of dissolution may be attributed at least in part to the fact that the energy required for dissolution of an amorphous drug is less than that required for the dissolution of a crystalline or microcrystalline solid phase.

To make this approach a practical reality in drug development, however, new engeneering techniques must be developed to stabilize amorphous drugs and counteract their tendency to undergo physical and chemical changes. One way of stabilizing the amorphous state of a drug involves forming solid solutions of the drug in polymeric matrices.

Drug solubility in polymeric matrices is an important physical property that affects the stability of drugs in amorphous formulations. This property is important for selecting appropriate polymers and designing formulations for the delivery of amorphous drugs. For example, it defines the upper limit of drug loading without risk of crystallization. Despite its importance, there has been no standard technique for measuring the solubility of a drug in a polymer. The difficulty largely arises from the high viscosity of polymers, which makes achieving solubility equilibrium difficult.

Vasanthavada et al. used moisture to induce the crystallization of trehalose from an amorphous mixture with dextran or PVP, and compared the eventual glass transition temperature Tg of the system with the Tgs of the amorphous mixtures of trehalose and polymer to calculate trehalose's solubility in the polymer (Vasanthavada, M.; Tong, W.; Joshi, Y.; Kislalioglu, M. S. Pharm. Res. 2004, 21, 1598-1606; Vasanthavada, M.; Tong, W.; Joshi, Y.; Kislalioglu, M. S. Pharm. Res. 2004, 22, 440-448). The method is subject to errors because of the effect of water on the solubility of drug in the polymer, and the effect of residual water on the Tg measurement.

Marsac et al. developed a predictive model of drug-polymer solubility based on the Flory-Huggins theory of liquids (Marsac, P. J.; Shamblin, S. L.; Taylor, L. S. Pharm. Res. 2006, 23, 2417-2426). The model has been calibrated on the solubility in a monomer solvent, but has never been tested with experimentally measured solubility in polymers.

Another difficulty encountered in studying drug/polymer solubility is the fact that pharmaceutically important drug/polymer dispersions are glasses, which are kinetically frozen liquids. Despite their low molecular mobility, glasses are characterized by their slow relaxation over time toward the equilibrium liquid state (the state that the system would reach if it were not kinetically frozen). This means that the solubility for a glassy drug/polymer system is not unique, and depends on the age of the mixture. The model of Marsac et al., and models based on equilibrium thermodynamics in general, predict solubility for fully relaxed, equilibrium liquids. In practice, however, structural relaxation of a glass is so slow that reaching the equilibrium liquid state may take years or even decades. For the shelf stability of pharmaceuticals, what is relevant is the solubility in the metastable glass, not the solubility in the equilibrium liquid. However, due to the low molecular mobility of glasses, measuring solubility in a glassy state is impractically slow.

DSC, a convenient technique available in most laboratories, has been used to measure the solubility of small-molecule crystals in small-molecule solvents (Mohan, R.; Lorenz, H.; Myerson, A. Ind. Eng. Chem. Res. 2002, 41, 4854-4862; Park, K.; Evans, J. M. B.; Myerson, A. Cryst. Growth & Des. 2003, 3, 991-995, Tamagawa R.; Martins, W.; Derenzo, S.; Bernardo, A.; Rolemberg, M.; Carvan, P.; Giulietti, M. Cryst. Growth & Des. 2006, 6(1), 313-320). The technique has also been applied in a method for evaluating the solubility of a crystalline substance in a polymer near the glass transition temperature (WO 2009/135799). It involves heating a crystal/solvent slurry of known composition x to slowly dissolve the crystals in the solvent and detecting the final temperature of crystal dissolution, T_{end}. If phase equilibrium is maintained during heating, the solubility of the crystal in the solvent is x at T_{end}.

Measuring solubilities in polymers is difficult because their high viscosity impedes the attainment of solubility equilibrium. This is of particular relevance for temperature near the glass transition temperature Tg of the system. The scanning method of WO 2009/135799 addressed this problem by measuring at different heating rates and extrapolating T_{end} to zero heating rate.

A major aim of the present invention is to provide an alternative method for measuring the solubility of a crystalline substance, in particular a pharmaceutically active ingredient, in a polymer with special concern to improving accuracy of measurement near the glass transition temperature. It has been found by the inventors that the method of the invention yields results consistent with those obtained with the scanning method of WO 2009/135799 at relatively high temperatures. Moreover, it revises slightly the results of the previous method at lower temperatures and extends the feasable temperature range of measurement to lower temperatures.

### SUMMARY OF THE INVENTION

This invention provides a method for evaluating the solubility of a crystalline substance in a polymer, comprising
a) providing at least one sample of an intimate crystalline substance/polymer mixture at a predetermined crystalline substance concentration;
b) annealing the sample at a constant temperature Tₐ for a period of time;
c) heating the annealed sample while recording the heat flux by DSC;
d) identifying a DSC dissolution endotherm in the recorded heat flux, if any;
e) considering the crystalline substance concentration as a concentration above the crystalline substance solubility in the polymer at temperature Tₐ when there is a DSC dissolution endotherm identified, and considering the crystalline substance concentration as a concentration less than or equal to the crystalline substance solubility in the polymer at temperature Tₐ when there is no DSC dissolution endotherm identified.

In one embodiment, the method comprises (a) providing a plurality of samples at different crystalline substance concentrations. For a plurality of samples at different crystalline substance concentrations annealed at a given temperature, the method would yield the upper and lower bounds for its equilibrium solubility at this temperature.

In another embodiment, the method comprises (a) providing a plurality of samples at the same crystalline substance concentration and (b) annealing the samples at different temperatures Tₐ For a crystalline substance/polymer mixture annealed at different temperature, the method would yield the upper and lower bounds for its equilibrium solubility temperature.

In a suitable embodiment, when there is a DSC dissolution endotherm identified, steps (a) to (e) are repeated at a lower crystalline substance concentration or at a higher temperature Tₐ Alternatively, when there is no DSC dissolution endotherm identified, steps (a) to (e) are repeated at a higher crystalline substance concentration or at a lower temperature Tₐ.

The invention further provides a method for evaluating the solubility of a crystalline substance in a polymer at the glass transition temperature Tg, comprising
a) establishing the solubility of the crystalline substance in the polymer as a function of temperature by the method described above,
b) providing a plurality of crystalline substance/polymer mixtures with different compositions and determining Tg of a liquid formed by melting each of the mixtures,
b) plotting Tg over the composition of the mixtures,
c) determining the solubility at Tg as the intersection of the solubility temperature plot and the Tg plot.

### BRIEF SUMMARY OF THE FIGURES

FIG. 1 shows a phase diagram for a drug-polymer system. Lines leading to e show different ways to reach solubility equilibrium and measure solubilities. Tm=melting point; Tg=glass transition temperature; w₁=drug weight fraction; w₂=polymer weight fraction

FIG. 2 shows the dissolution endotherms of 50% w/w nifedipine in vinyl pyrrolidone-vinyl acetate copolymer prepared by cryomilling and annealed for 60 min at the temperatures indicated.

FIG. 3 shows a comparison of the final temperature of crystal dissolution, T_{end}, obtained with the scanning method of WO 2009/135799 (circles) and annealing method of the present invention (crosses) for three systems.

FIG. 4 shows the solute activity of indomethacin or nifedipine a₁ vs. polymer weight fraction w₂. The solid curves are Flory-Huggins fits.

### DETAILED DESCRIPTION OF THE INVENTION

The term “crystalline substance concentration” as used herein refers to the weight of the crystalline substance, relative to the combined weight of the crystalline substance and the polymer, for example expressed as a percentage. Suitably, the composition may be within the range from 1% to 99% w/w of the crystalline substance, for example 5% to 95%.

In the thermodynamic sense, measuring solubility means determining the temperature and the solution concentration at which a system achieves equilibrium. In reference to the binary phase diagram shown in FIG. 1, the goal is to determine the coordinate (T, w) of a solubility equilibrium e, where T is temperature and w is concentration. One can measure solubility in different ways according to how solubility equilibrium is approached: (1) Follow the increase of solution concentration at constant T as the solute dissolves into an under-saturated solution (path ae); (2) Follow the decrease of solution concentration at constant T as the solute crystallizes from a super-saturated solution (path be); (3) Measure the solution temperature or depressed melting point for a solute-solvent physical mixture of concentration w (path ce); (4) Measure the crystallization temperature or depressed freezing point for a saturated solution of concentration w (path de). Besides the solubility curve, FIG. 1 also shows a glass transition temperature vs. concentration curve. This is a reminder that for typical drug-polymer dispersions, solubility values often need to be measured near the glass transition temperatures, at which the rates of approaching solubility equilibrium would be especially low.

The method of the invention can be used to approach the solution equilibrium e by varying the annealing temperature of samples of a given crystalline substance concentration, e.g. starting at a lower (c) or higher (d) temperature and iteratively approaching e. Alternatively, the solution equilibrium e may be approached by varying the crystalline substance concentration of the samples while annealing at the same temperature, e.g. starting at a lower (a) or higher (b) crystalline substance concentration and iteratively approaching e.

It is advantageous that Tₐ is chosen to be at or near an estimated solution temperature. This estimated solution temperature may be determined by any method known in the art, for example by the scanning method of WO 2009/135799. Thus, the effort of determining the solubility of a crystalline substance in a polymer may be reduced. Using the method of the invention more accurate data for estimated solution temperature values can be obtained.

In the invention, a DSC method is used to identify the presence of a dissolution endotherm, i.e. to determine whether undissolved crystals still remain.

Differential scanning calorimetry, or DSC, is a thermoanalytical technique in which the difference in the amount of heat required to increase the temperature of a sample and a reference are measured as a function of temperature, i.e. in which the heat flux over time of a sample is recorded. Both the sample and the reference are maintained at nearly the same temperature throughout the experiment. Generally, the temperature program for a DSC analysis is designed such that the sample holder temperature increases linearly as a function of time. The reference sample has a well-defined heat capacity over the range of temperatures to be scanned. The basic principle underlying this technique is that, when the sample undergoes a physical transformation such as phase transitions, more (or less) heat will need to flow to it than to the reference to maintain both at the same temperature. Whether more or less heat is needed flow to the sample depends on whether the process is exothermic or endothermic.

As the crystalline substance dissolves in the polymer, the sample generally undergoes an endothermic phase transition. In the DSC endotherm, a corresponding transition peak will be observed.

Because dissolution of the drug in the polymer matrix requires the transport of materials, how well the components are mixed affects the kinetics of dissolution. If the components are poorly mixed and contain large particles, dissolution requires material transport over long distances. When this mixture is held at a specific annealing temperature, the annealing time will be much longer. Accordingly, the mixture of the crystalline substance and the polymer should be as intimate as possible without, however, significantly losing crystallinity.

Various milling or grinding techniques known in the art may be employed, such as hand milling, ball milling and the like. The frictional heat generated in conventional dry grinding may be detrimental to retaining crystallinity. Therefore, controlling the temperature during milling is preferable. We found that cryomilling (also referred to as cryogenic milling, cryogenic grinding or freezer milling) the drug/polymer mixture before DSC is an effective way to improve mixing and help achieve solubility equilibrium. Cryomilling is a variation of mechanical milling, in which a powder is milled in a cryogen (usually liquid nitrogen) slurry or at a cryogenic temperature.

In preferred embodiments, the intimate mixture is obtained by joint cryomilling of the crystalline substance and the polymer. Suitable cryomilling times range from 1 min to 60 min. An optimal time of cryomilling can be determined by increasing the milling time until the point of diminishing return. The crystalline substance and the polymer may also be milled separately, combined, and further cryomilled together.

To ensure that a sample reached phase equilibrium, it is kept (annealed) at a constant annealing temperature Tₐ for a period of time prior to measurement. The annealing time is selected in a manner that the whole sample reaches phase equilibrium within this period. In general, the closer the temperature of a sample is to the glass transitions temperature, the higher is its viscosity, and the longer it will take it to reach phase equilibrium. A suitable annealing time may be established by annealing identical samples for successively increasing annealing times and investigating the residual endotherm. When the residual endotherm reaches a plateau value (i.e. does not dimish upon further increase of annealing time) or reaches zero, the annealing time is sufficient for the sample to reach equilibrium. The annealing time may vary, e.g., from 30 min to several hours, mostly 30 min to 2 hours. An annealing time of at least 60 min is suitable in most instances.

After the annealing period the sample is scanned by DSC to determine whether undissolved crystals still remain. This involves brief cooling of the sample and subjecting the sample to DSC scanning over a temperature range including the annealing temperature Tₐ. DSC heating rates in the range of 50° C./min to 2° C./min, preferably 20° C./min to 5° C./min, for example 10° C./min are generally suitable. The use of a relatively high heating rate improves the sensitivity of detecting residual crystals.

For the purposes of the present invention, the crystalline substance/polymer solubility near the glass transition temperature is of particular interest. The glass transition temperature Tg of the pure polymer is generally known. Dissolved substances in the polymer, however, could exert a plasticizing or anti-plasticizing effect on the polymer and thus depress or elevate the Tg of the polymer such that the crystalline substance/polymer solid solution has a somewhat lower or higher Tg than the starting polymer used for its preparation. Accordingly, the Tg depends on the composition of the mixture. The Tg—composition relation can be established by determining the Tg associated with homogeneous amorphous substance/polymer mixtures having different compositions, and plotting Tg over the composition of the mixtures.

The Tg associated with a homogeneous amorphous drug/polymer mixture is conveniently determined by a DSC method, in particular by modulated DSC (MDSC). In contrast to DSC, which measures heat flow as a function of a constant rate of change in temperature, modulated DSC superimposes a sinusoidal temperature modulation on this rate which permits to measure the heat-capacity effects simultaneously with the kinetic effect. The glass transition is observed as a step-like change in the DSC curve, which results from the increase of heat capacity when a solid glass is heated to become a viscous liquid.

The solubility of the crystalline substance in the polymer at Tg can be regarded as an upper concentration limit. A solid solution whose crystalline substance concentration is below this concentration limit is assigned as likely stable against crystallization. If the crystalline substance concentration of a solid solution exceeds the upper concentration limit, it should be kept at temperatures below Tg of the solid solution.

In addition to examining physical stability of amorphous solid solutions and identifying the safe storage conditions for such formulations, the upper concentration limit (UCL) estimated for pairs of crystalline substances and polymers can be further utilized in designing and optimizing formulations of amorphous solid solutions, also from the physical stability viewpoint. Different polymers could be used for formulation development. Various auxiliary components, such as surfactants, could be added to the formulation as well.

The crystalline substance may be any chemical substance of interest that is present in its crystalline state. In preferred embodiments, however, the crystalline substance is a pharmaceutically active ingredient (drug). Pharmaceutically active ingredients are biologically active agents and include those which exert a local physiological effect, as well as those which exert a systemic effect, after oral administration. The invention is particularly useful for water-insoluble or poorly water-soluble (or “hydrophobic” or “lipophilic”) compounds. Compounds are considered water-insoluble or poorly water-soluble when their solubility in water at 25° C. is less than 1 g/100 ml, especially less than 0.1 g/100 ml.

Examples of suitable pharmaceutically active ingredients include, but are not limited to:

analgesics and anti-inflammatory drugs such as fentanyl, indomethacin, ibuprofen, naproxene, diclofenac, diclofenac sodium, fenoprofen, acetylsalicylic acid, ketoprofen, nabumetone, paracetamol, piroxicam, meloxicam, tramadol, and COX-2 inhibitors such as celecoxib and rofecoxib;
anti-arrhythmic drugs such as procainamide, quinidine and verapamil;
antibacterial and antiprotozoal agents such as amoxicillin, ampicillin, benzathine penicillin, benzylpenicillin, cefaclor, cefadroxil, cefprozil, cefuroxime axetil, cephalexin, chloramphenicol, chloroquine, ciprofloxacin, clarithromycin, clavulanic acid, clindamycin, doxyxycline, erythromycin, flucloxacillin sodium, halofantrine, isoniazid, kanamycin sulphate, lincomycin, mefloquine, minocycline, nafcillin sodium, nalidixic acid, neomycin, nortloxacin, ofloxacin, oxacillin, phenoxymethyl-penicillin potassium, pyrimethamine-sulfadoxime and streptomycin;
anti-coagulants such as warfarin;
antidepressants such as amitriptyline, amoxapine, butriptyline, clomipramine, desipramine, dothiepin, doxepin, fluoxetine, reboxetine, amineptine, selegiline, gepirone, imipramine, lithium carbonate, mianserin, milnacipran, nortriptyline, paroxetine, sertraline and 3-[2-[3,4-dihydrobenzofuro[3,2-c]pyridin-2(1H)-yl]ethyl]-2-methyl-4H-pyrido[1,2-a]pyrimidin-4-one;
anti-diabetic drugs such as glibenclamide and metformin;
anti-epileptic drugs such as carbamazepine, clonazepam, ethosuximide, gabapentin, lamotrigine, levetiracetam, phenobarbitone, phenyloin, primidone, tiagabine, topiramate, valpromide and vigabatrin;
antifungal agents such as amphotericin, clotrimazole, econazole, fluconazole, flucytosine, griseofulvin, itraconazole, ketoconazole, miconazole nitrate, nystatin, terbinafine and voriconazole;
antihistamines such as astemizole, cinnarizine, cyproheptadine, decarboethoxyloratadine, fexofenadine, flunarizine, levocabastine, loratadine, norastemizole, oxatomide, promethazine and terfenadine;
anti-hypertensive drugs such as captopril, enalapril, ketanserin, lisinopril, minoxidil, prazosin, ramipril, reserpine, terazosin and telmisartan;
anti-muscarinic agents such as atropine sulphate and hyoscine;
antineoplastic agents and antimetabolites such as platinum compounds, such as cisplatin and carboplatin; taxanes such as paclitaxel and docetaxel; tecans such as camptothecin, irinotecan and topotecan; vinca alkaloids such as vinblastine, vindecine, vincristine and vinorelbine; nucleoside derivatives and folic acid antagonists such as 5-fluorouracil, capecitabine, gemcitabine, mercaptopurine, thioguanine, cladribine and methotrexate; alkylating agents such as the nitrogen mustards, e.g. cyclophosphamide, chlorambucil, chiormethine, iphosphamide, melphalan, or the nitrosoureas, e.g. carmustine, lomustine, or other alkylating agents, e.g. busulphan, dacarbazine, procarbazine, thiotepa; antibiotics such as daunorubicin, doxorubicin, idarubicin, epirubicin, bleomycin, dactinomycin and mitomycin; HER 2 antibodies such as trastuzumab; podophyllotoxin derivatives such as etoposide and teniposide; farnesyl transferase inhibitors; anthrachinon derivatives such as mitoxantron;
anti-migraine drugs such as alniditan, naratriptan and sumatriptan;
anti-Parkinsonian drugs such as bromocryptine mesylate, levodopa and selegiline;
antipsychotic, hypnotic and sedating agents such as alprazolam, buspirone, chlordiazepoxide, chlorpromazine, clozapine, diazepam, flupenthixol, fluphenazine, flurazepam, 9-hydroxyrisperidone, lorazepam, mazapertine, olanzapine, oxazepam, pimozide, pipamperone, piracetam, promazine, risperidone, selfotel, seroquel, sertindole, sulpiride, temazepam, thiothixene, triazolam, trifluperidol, ziprasidone and zolpidem;
anti-stroke agents such as lubeluzole, lubeluzole oxide, riluzole, aptiganel, eliprodil and remacemide;
antitussives such as dextromethorphan and laevodropropizine;
antivirals such as acyclovir, ganciclovir, loviride, tivirapine, zidovudine, lamivudine, zidovudine/lamivudine, didanosine, zalcitabine, stavudine, abacavir, lopinavir, amprenavir, nevirapine, efavirenz, delavirdine, indinavir, nelfinavir, ritonavir, saquinavir, adefovir and hydroxyurea;
beta-adrenoceptor blocking agents such as atenolol, carvedilol, metoprolol, nebivolol and propanolol;
cardiac inotropic agents such as aminone, digitoxin, digoxin and milrinone;
corticosteroids such as beclomethasone dipropionate, betamethasone, budesonide, dexamethasone, hydrocortisone, methylprednisolone, prednisolone, prednisone and triamcinolone;
disinfectants such as chlorhexidine;
diuretics such as acetazolamide, furosemide, hydrochlorothiazide and isosorbide;
enzymes;
gastro-intestinal agents such as cimetidine, cisapride, clebopride, diphenoxylate, domperidone, famotidine, lansoprazole, loperamide, loperamide oxide, mesalazine, metoclopramide, mosapride, nizatidine, norcisapride, olsalazine, omeprazole, pantoprazole, perprazole, prucalopride, rabeprazole, ranitidine, ridogrel and sulphasalazine;
haemostatics such as aminocaproic acid;
HIV protease inhibiting compounds such as ritonavir, lopinavir, indinavir, saquinavir, 5(S)-Boc-amino-4(S)-hydroxy-6-phenyl-2(R)phenylmethylhexanoyl-(L)-Val-(L)-Phe-morpholin-4-ylamide, 1-Naphthoxyacetyl-beta-methylthio-Ala-(2S,3S)3-amino-2-hydroxy-4-butanoyl 1,3-thiazolidine-4-t-butylamide, 5-isoquinolinoxyacetyl-beta-methylthio-Ala-(2S,3S)-3-amino-2-hydroxy-4-butanoyl-1,3-thiazolidine-4-t-butylamide, [1S-[1R—(R-),2S*])—N′-[3-[[[(1,1-dimethylethyl)amino]carbonyl](2-methylpropyl)amino]-2hydroxy-1-(phenylmethyl)propyl]-2-[(2-quinolinylcarbonyl)amino]-butanediamide, amprenavir; DMP-323; DMP-450; nelfinavir, atazanavir, tipranavir, palinavir, darunavir, RO033-4649, fosamprenavir, P-1946, BMS 186,318, SC-55389a; BILA 1906 BS, tipranavir;
lipid regulating agents such as atorvastatin, fenofibrate, fenofibric acid, lovastatin, pravastatin, probucol and simvastatin;
local anaesthetics such as benzocaine and lignocaine;
opioid analgesics such as buprenorphine, codeine, dextromoramide, dihydrocodeine, hydrocodone, oxycodone and morphine;
parasympathomimetics and anti-dementia drugs such as AIT-082, eptastigmine, galanthamine, metrifonate, milameline, neostigmine, physostigmine, tacrine, donepezil, rivastigmine, sabcomeline, talsaclidine, xanomeline, memantine and lazabemide;
peptides and proteins such as antibodies, becaplermin, cyclosporine, tacrolimus, erythropoietin, immunoglobulins and insuline;
sex hormones such as oestrogens: conjugated oestrogens, ethinyloestradiol, mestranol, oestradiol, oestriol, oestrone; progestogens; chlormadinone acetate, cyproterone acetate, 17-deacetyl norgestimate, desogestrel, dienogest, dydrogesterone, ethynodiol diacetate, gestodene, 3-keto desogestrel, levonorgestrel, lynestrenol, medroxy-progesterone acetate, megestrol, norethindrone, norethindrone acetate, norethisterone, norethisterone acetate, norethynodrel, norgestimate, norgestrel, norgestrienone, progesterone and quingestanol acetate;
stimulating agents such as sildenafil, vardenafil;
vasodilators such as amlodipine, buflomedil, amyl nitrite, diltiazem, dipyridamole, glyceryl trinitrate, isosorbide dinitrate, lidoflazine, molsidomine, nicardipine, nifedipine, oxpentifylline and pentaerythritol tetranitrate;
their N-oxides, their pharmaceutically acceptable acid or base addition salts and their stepreochemically isomeric forms.

Pharmaceutically acceptable acid addition salts comprise the acid addition salt forms which can be conveniently obtained by treating the base form of the active ingredient with appropriate organic and inorganic acids.

Active ingredients containing an acidic proton may be converted into their non-toxic metal or amine addition salt forms by treatment with appropriate organic and inorganic bases.

The term “addition salt” also comprises the hydrates and solvent addition forms which the active ingredients are able to form. Examples of such forms are hydrates, alcoholates and the like.

The N-oxide forms of the active ingredients comprise those active ingredients in which one or several nitrogen atoms are oxidized to the so-called N-oxide.

The term “stereochemically isomeric forms” defines all possible stereoisomeric forms which the active ingredients may possess. In particular, stereogenic centers may have the R- or S-configuration and active ingredients containing one or more double bonds may have the E- or Z-configuration.

The polymer may be any polymeric matter of interest. For the envisaged use of the solid solution as a dosage form for the delivery of a pharmaceutically active ingredient to a subject, in particular a human, the polymer is, however, preferably a pharmaceutically acceptable polymer.

The pharmaceutically acceptable polymer may be selected from water-soluble polymers, water-dispersible polymers or water-swellable polymers or any mixture thereof. Polymers are considered water-soluble if they form a clear homogeneous solution in water. When dissolved at 20° C. in an aqueous solution at 2% (w/v), the water-soluble polymer preferably has an apparent viscosity of 1 to 5000 mPa·s, more preferably of 1 to 700 mPa·s, and most preferably of 5 to 100 mPa·s. Water-dispersible polymers are those that, when contacted with water, form colloidal dispersions rather than a clear solution. Upon contact with water or aqueous solutions, water-swellable polymers typically form a rubbery gel.

Preferably, the pharmaceutically acceptable polymer has a Tg of at least 40° C., preferably at least 50° C., most preferably from 80° to 180° C. “Tg” means glass transition temperature. Methods for determining the Tg values of organic polymers are described in “Introduction to Physical Polymer Science”, 2^{nd} Edition by L. H. Sperling, published by John Wiley & Sons, Inc., 1992. The Tg value can be calculated as the weighted sum of the Tg values for homopolymers derived from each of the individual monomers, i, that make up the polymer: Tg=Σ Wi Xi, where W is the weight percent of monomer i in the organic polymer, and X is the Tg value for the homopolymer derived from monomer i. The Tg values for the homopolymers may be taken from “Polymer Handbook”, 2^{nd} Edition by J. Brandrup and E. H. Immergut, Editors, published by John Wiley & Sons, Inc., 1975.

For example, preferred pharmaceutically acceptable polymers can be selected from homopolymers of N-vinyl lactams, especially polyvinylpyrrolidone (PVP); different grades of commercially available PVP are PVP K-12, PVP K-15, PVP K-17, PVP K-20, PVP K-25, PVP K-30, PVP K-60, PVP K-90 and PVP K-120. The K-value referred to in this nomenclature is calculated by Fikentscher's formula from the viscosity of the PVP in aqueous solution, relative to that of water;

copolymers of N-vinyl lactams, especially copolymers of N-vinyl pyrrolidone and vinyl acetate or copolymers of N-vinyl pyrrolidone and vinyl propionate,
cellulose esters and cellulose ethers, in particular methylcellulose and ethylcellulose, hydroxyalkylcelluloses, in particular hydroxypropylcellulose, hydroxyalkylalkylcelluloses, in particular hydroxypropylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate;
high molecular polyalkylene oxides such as polyethylene oxide and polypropylene oxide and copolymers of ethylene oxide and propylene oxide,
polyvinyl alcohol-polyethylene glycol-graft copolymers (available as Kollicoat® IR from BASF AG, Ludwigshafen, Germany);
polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates),
polyacrylamides,
vinyl acetate polymers such as copolymers of vinyl acetate and crotonic acid, partially hydrolyzed polyvinyl acetate (also referred to as partially saponified “polyvinyl alcohol”),
polyvinyl alcohol,
oligo- and polysaccharides such as carrageenans, galactomannans and xanthan gum, or mixtures of one or more thereof.

Among these, homopolymers or copolymers of N-vinyl pyrrolidone, in particular a copolymer of N-vinyl pyrrolidone and vinyl acetate, are preferred. A particularly preferred polymer is a copolymer of 60% by weight of the copolymer, N-vinyl pyrrolidone and 40% by weight of the copolymer, vinyl acetate.

The invention is illustrated by the appended drawings and the examples which follow.

### EXAMPLES

D-mannitol (99+%, β polymorph), indomethacin (IMC, γ polymorph), a nonsteroidal anti-inflammatory agent, and nifedipine (NIF, α polymorph), a calcium channel blocker, were obtained from Sigma-Aldrich. Polyvinyl pyrrolidone (PVP) K-12 (MW ˜2,500, 5% moisture), K-15 (MW ˜8,000, 12% w/w moisture) and K-25 (MW 28,000-34,000, 8% w/w moisture) were obtained from BASF. Polyvinyl acetate (MW 83,000) was obtained from Sigma-Aldrich. The different molecular-weight grades of PVP were used to evaluate the dependence of solubility on the molecular weight of the polymer. PVP/VA being a 60:40 vinyl pyrrolidone-vinyl acetate copolymer (Kollidone VA64, MW 45,000-70,000, 5% w/w moisture, Tg 101° C.) was supplied by Abbott. PVAc (MW ˜83,000, 2% w/w moisture) was obtained from Sigma-Aldrich.

Solute-polymer mixtures of desired concentrations were prepared by weighing the components. Each concentration was corrected for the amount of water absorbed by the polymer; as a result, what is referred to below as, e.g., 50% w/w NIF in PVP/VA actually contained 51.3% w/w NIF in PVP/VA on the dry basis. A cryogenic impact mill (SPEX CertiPrep model 6750) with liquid nitrogen as coolant was used to prepare solute/polymer mixtures of different concentrations. In a typical procedure, 0.5-1 g solute/polymer powder was milled at 10 Hz. Each cycle of milling was 2 min, followed by a 2 min cool-down. The cycle was repeated to achieve a desired milling time (2-60 min). For all materials used for solubility measurement, analysis by X-ray powder diffraction confirmed that the crystals remaining after milling were of the original polymorph of the respective material. X-ray diffraction was performed with a Bruker D8 Advance diffractometer (Cu Kα radiation, voltage 40 kV, and current 40 mA). The sample was ground, if necessary, with mortar and pestle, placed on a zero-background silicon (510) sample holder, and scanned from 2° to 50° (20) at a speed of 1° C./min and a step size of 0.02°.

For annealing, 5-10 mg of sample was packed into a Tzero hermetic aluminum pan with three pin holes made in the lid to allow the escape of moisture and held isothermally at a desired temperature for as long as 600 minutes. Shorter annealing time was used if the system could apparently achieve equilibrium sooner.

Differential scanning calorimetry (DSC) was conducted using a TA Instruments DSC Q2000 at 10° C./min to determine whether residual crystals remain after annealing, i.e. whether a dissolution endotherm can be identified. Unless otherwise noted, the reported Tg is the onset of the glass transition.

### Example 1

### Dissolution of NIF in PVP/VA

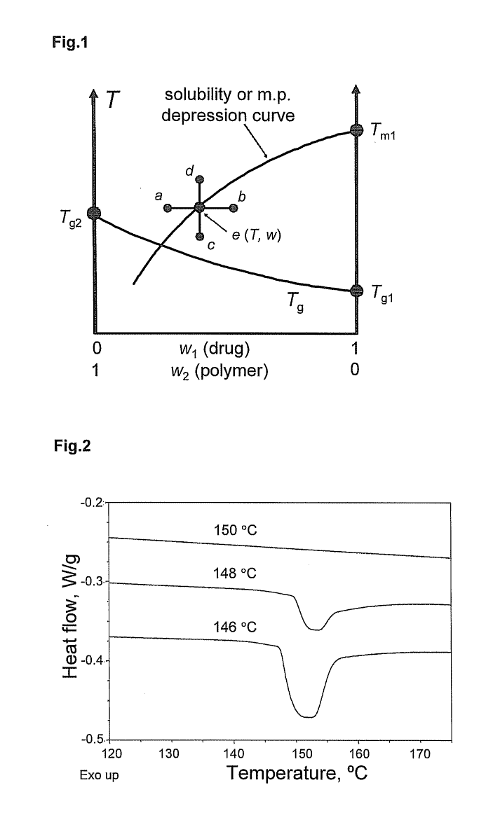

A mixture containing 50% w/w NIF in PVP/VA prepared by cryo-milling was annealed at 150, 148, and 146° C. for 60° min and scanned at 10° C./min to determine whether any crystals still remained after annealing. FIG. 2 shows that annealing at 150° C. led to full dissolution (no dissolution endotherm identifiable), whereas annealing at or below 148° C. did not. Assuming attainment of phase equilibrium, these results indicate that the solubility temperature for this mixture is between 148 and 150° C., which agrees well with T_{end} of 150° C. determined by the method provided by WO 2009/135799.

### Example 2

### Solubility of D-Mannitol in PVP, NIF in PVP/VA and IMC in PVP/VA

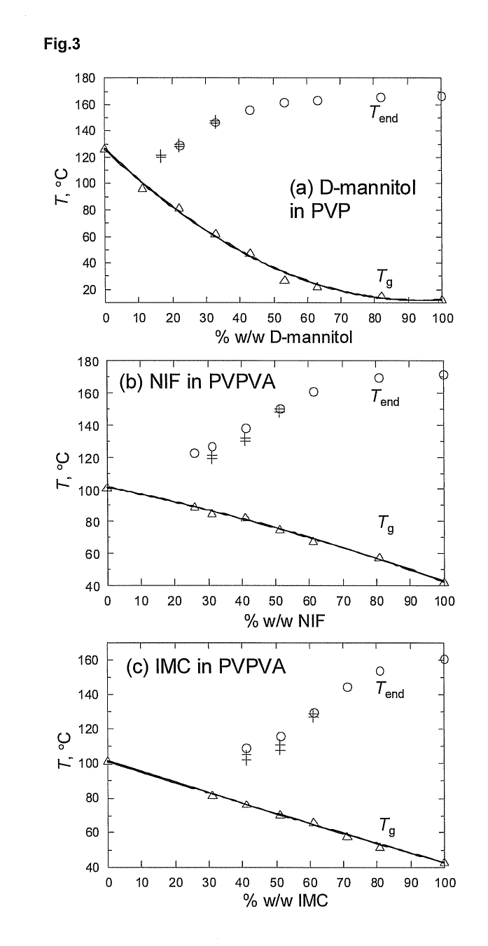

FIG. 3 shows T_{end} values of mixtures of D-mannitol in PVP K-15, of NIF in PVP/VA and of IMC in PVP/VA, which were determined by the annealing method of the present invention (crosses) and by the scanning method provided by WO 2009/135799 (circles). The two crosses at each concentration are the upper and lower limits of T_{end}. Additionally, the curves of glass transition temperatures Tg (triangles) are given in FIG. 3. For the D-mannitol-PVP mixtures, Tg is the inflection point because the onset temperature is difficult to define at high polymer concentrations. For the other mixtures, Tg is the onset temperature.

For D-mannitol dissolving in PVP K15, the results of both methods were consistent. However, the annealing method slightly lowered the temperature of measurement, closer to the glass transition temperature (FIG. 3 a). Whereas the scanning method could be performed only down to ca. 20% w/w D-mannitol, the annealing method could be performed at 15% w/w, for which T_{end} was found to be between 115 and 120° C.

For the mixtures of NIF in PVP/VA and IMC in PVP/VA, the annealing method yielded results consistent with those obtained with the scanning method at higher temperatures, but revised slightly the results at lower temperatures (FIGS. 3 b and c). For 30 and 40% w/w NIF in PVP/VA, the annealing method yielded solution temperatures approximately 7° C. below those obtained with the scanning method of WO 2009/135799. The same observation was made for 40 and 50% w/w IMC in PVP/VA.

The greater difference between the results of the scanning and annealing method at lower drug concentrations (FIGS. 3 b and c) may be explained by the equilibrium solubility temperature approaching the glass transition temperature of the saturated solution. If dissolution takes place in a highly viscous system, solubility equilibrium may be too slow to establish during the time scale of DSC scanning. Because the annealing method allows for longer equilibration times, its results should be more accurate.

### Example 3

### Comparison of Two Variants of the Annealing Method which Vary the Concentration and the Annealing Temperature, Respectively

In an alternative annealing scheme, mixtures of different concentrations were held at the same temperature to determine the lower and upper bounds for the solubility at the annealing temperature. Using variant of the annealing method, the solubility of D-mannitol in PVP K15 was determined to be between 20 and 30% w/w at 130° C., the solubility of NIF in PVP/VA to be between 30 to 40% w/w at 123° C., and the solubility of IMC in PVP/VA to be between 50 to 60% w/w at 110° C. These results are consistent with those of the annealing method as performed in example 2, where samples of a defined concentration were annealed at varying temperatures.

### Example 4

### Comparison of Solute Activity Determined by Different Methods

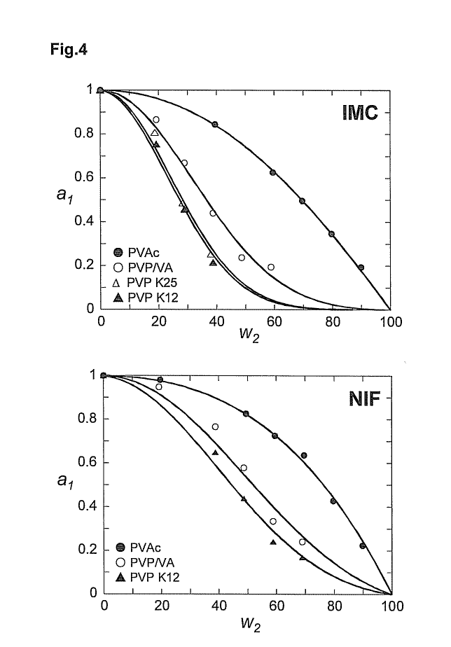

The method of the present invention was used to determine solubility data of IMC and NIF in PVP K-12. The solute activity a₁ of IMC or NIF was calculated and plotted against the polymer weight fraction w₂ (FIG. 4). The solute activity a₁ of the drug in the saturated solution was calculated from the solubility of the crystalline drug in the polymer as follows:

In a₁=(ΔHₘ/R)(1/Tₘ−1/T) (1)

where Tₘ is the melting point of the pure drug, ΔHₘ is its molar heat of melting, and T is the temperature at which the drug's solubility is measured (or its depressed melting point). As shown in FIG. 4, the solute activity decreases with the increase of polymer weight fraction w₂. According to the Flory-Huggins theory, the solute activity is given by

In a₁=In v₁+(1−1/x)v2+χv₂² (2)

where v₁ and v₂ are the solute (drug) and solvent (polymer) volume fractions (v₁+v₂=1), x is the molar volume ratio of the polymer and the drug, and χ□ is the solute-solvent interaction parameter. The solid curves in FIG. 4 are the results of fitting the activities to the Flory-Huggins model. In this analysis, we have assumed that the volume fraction is the same as the weight fraction and the parameter x is the ratio of the molecular weights of the polymer and the drug.

The Flory-Huggins model fits the data in FIG. 4 reasonably well. To the extent the reasonable fitting in FIG. 4 justifies the use of the Flory-Huggins model, the interaction parameter χ (equation 1) could be obtained.

TABLE 1
Drug-polymer interaction parameters.

| | | solute-solvent interaction parameter χ as determined | |
|---|---|---|---|
| | | with the method of the | |
| Drug | Polymer | present invention | by Marsac et al.* |
| NIF | PVP K-12 | −2.5 ± 0.2 | 0.0 |
| IMC | PVP K-12 | −8.2 ± 0.6 | −0.82 |

*Marsac, P. J.; Shamblin, S. L.; Taylor, L. S. Pharm. Res. 2006, 23, 2417-2426

The χ values thus obtained are substantially more negative (indicating stronger attractive interactions) from those of Marsac et al. (Table 1). This difference probably arises from the different experimental conditions of the two studies. Marsac et al. used a scanning method to measure polymer-depressed melting points of crystalline drug; their sample was a physical mixture of a drug and a polymer (or a drug and a drug-polymer dispersion); their DSC scan rate was 1° C./min. In the method of the present invention, cryo-milling is used to improve the uniformity and reduce the particle size of drug-polymer mixtures so that solubility equilibrium is more easily achieved; a long equilibration (annealing) time is provided to improve the likelihood of achieving solubility equilibrium. Together, these measures likely have provided more accurate results, especially at lower temperatures.

1. A method for evaluating the solubility of a crystalline substance in a polymer, comprising
a) providing at least one sample of an intimate crystalline substance/polymer mixture at a predetermined crystalline substance concentration;
b) annealing the sample at a constant temperature Tₐ for a period of time;
c) heating the annealed sample while recording the heat flux over time by DSC;
d) identifying a DSC dissolution endotherm in the recorded heat flux, if any;
e) considering the crystalline substance concentration as a concentration above the crystalline substance solubility in the polymer at temperature Tₐ when there is a DSC dissolution endotherm identified, and considering the crystalline substance concentration as a concentration less than or equal to the crystalline substance solubility in the polymer at temperature Tₐ when there is no DSC dissolution endotherm identified.

2. The method of claim 1, wherein the period of time for annealing the sample is at least 60 min.

3. The method of claim 1, which comprises (a) providing a plurality of samples at different crystalline substance concentrations.

4. The method of claim 1, which comprises (a) providing a plurality of samples at the same crystalline substance concentration and (b) annealing the samples at different temperatures Tₐ.

5. The method of claim 1, wherein, when there is a DSC dissolution endotherm identified, steps (a) to (e) are repeated at a lower crystalline substance concentration or at a higher temperature Tₐ.

6. The method of claim 1, wherein, when there is no DSC dissolution endotherm identified, steps (a) to (e) are repeated at a higher crystalline substance concentration or at a lower temperature Tₐ.

7. The method of claim 1, comprising recording the heat flux over time by DSC at a heating rate in the range of 5° C./min to 15° C./min.

8. The method of claim 1, wherein the intimate mixture is obtained by joint cryomilling of the crystalline substance and the polymer.

9. The method of any one of claim 1, wherein the crystalline substance is a pharmaceutically active ingredient.

10. A method for evaluating the solubility of a crystalline substance in a polymer at the glass transition temperature Tg, comprising
a) establishing the solubility of the crystalline substance in the polymer as a function of temperature by the method of claim 1,
b) providing a plurality of crystalline substance/polymer mixtures having different crystalline substance concentrations and determining Tg of a liquid formed by melting each of the mixtures,
b) plotting Tg over the crystalline substance concentrations of the mixtures,
c) determining the solubility at Tg as the intersection of the solubility temperature plot and the Tg plot.

11. The method of claim 10, wherein Tg is determined by a DSC method.
